(19) United States
(12) Patent Application Publication
(10) Pub. No.: US 2015/0314129 A1
(43) Pub. Date: Nov. 5, 2015

(54) HERMETIC HOUSING, PARTICULARLY FOR ENCAPSULATING AN IMPLANTABLE MEDICAL DEVICE

(71) Applicant: COMMISSARIAT A L'ENERGIE ATOMIQUE ET AUX ENERGIES ALTERNATIVES, Paris (FR)

(72) Inventors: Simon PERRAUD, Bandol (FR); Fabrice EMIEUX, Voreppe (FR); Nicolas KARST, Folkling (FR); Cecile LE COADOU, Grenoble (FR)

(21) Appl. No.: 14/650,016

(22) PCT Filed: Dec. 12, 2013
(86) PCT No.: PCT/EP2013/076353

§ 371 Date: Jun. 5, 2015

(30) Foreign Application Priority Data
Dec. 14, 2012 (FR) 1262075

(51) Int. Cl. A61N 1/375; H05K 5/00

(52) U.S. Cl. CPC A61N 1/375; H05K 5/0095

(57) ABSTRACT

Hermetic housing comprising: a first element (11) in sheet form, made from at least one material chosen from a metal, a ceramic and a glass; a second element (15, 17, 18) for covering said first element, likewise made of at least one material chosen from a metal, a ceramic and a glass; a first metal surround (12) interposed between said first element and said second element, comprising an internal part (14) partially or fully positioned within the perimeter of said first element and an outer part (13) positioned fully on the outside of said perimeter of said first element; a first hermetic seal between said first element and said internal part of said first metal surround; and a second hermetic seal between said second element and said outer part of said first metal surround. Method of encapsulating a device, notably an implantable medical device, using such a hermetic housing.

The invention relates to a hermetic or leaktight housing, preferably ultrathin and/or shapeable, which can in particular be suitable for the encapsulation of a device and more particularly but not exclusively of an implantable medical device. The invention also relates to a process for the encapsulation of a device, and in particular of an implantable medical device.

Implantable medical devices, such as pacemakers, cardiac defibrillators, heart monitors, neurostimulators, pumps, biomedical sensors, and the like, are generally encapsulated in a biocompatible metal housing, typically made of titanium, which is hermetically closed by laser beam welding. This type of encapsulation provides excellent hermeticity, in order, on the one hand, to protect the encapsulated components from attacks by biological tissues or fluids and, on the other hand, to protect the body of the patient from possible encapsulated elements which are toxic or bioincompatible. Glass-metal or ceramic-metal feedthroughs are made in the walls of the housing, in order to make possible the delivery of one or more electrodes while retaining perfect hermeticity. Such metal housings are well suited to encapsulating implantable medical devices existing in the form of bulky (with a typical thickness of between 5 and 20 mm) and mechanically rigid objects.

An alternative solution for the encapsulation of implantable medical devices consists in assembling a cap (that is to say, a part whose shape resembles that of a hat) on a substrate (that is to say, a flat part), as described, for example, in the document U.S. Pat. No. 5,750,926 and in the paper by A. Vanhoestenberghe et al., “Hermetic Encapsulation of an Implantable Vision Prosthesis—Combining Implant Fabrication Philosophies” (Proceedings of IFESS 2008, 21-25 Sep. 2008, Freiburg). In the document U.S. Pat. No. 5,750,926, the housing consists of a metal cap attached to an electrically insulating substrate; the hermeticity between the cap and the substrate can be provided by forming a first hermetic seal between a metal surround and the substrate and then a second hermetic seal by laser beam welding between the metal surround and the cap. In the abovementioned paper by A. Vanhoestenberghe et al., the housing consists of a ceramic cap attached to a ceramic substrate; the hermeticity between the cap and the substrate is provided by forming a first hermetic seal by soldering between a titanium surround and the substrate, a second hermetic seal by soldering between another titanium surround and the cap, and then a third hermetic seal by laser beam welding between the two titanium surrounds. In these two documents, the hermetic delivery of one or more electrodes is carried out via metal tracks and vertical metal feedthroughs.

The encapsulation methods described by these documents are well suited to obtaining devices after encapsulation which are thin (with a typical thickness of a few millimeters) and mechanically rigid, as demonstrated experimentally in the paper by Vanhoestenberghe et al. It is also possible to envisage using this type of encapsulation in order to obtain devices after encapsulation which are ultrathin (with a thickness of less than 3 mm), as described in the abovementioned document U.S. Pat. No. 5,750,926. However, in practice, it is technically difficult to reduce the thickness of the device after encapsulation under the bar of 3 mm. In particular, the use of ultrathin metal surrounds renders extremely problematic the formation of the final hermetic seal by laser beam welding as, if the surrounds are excessively thin, then the stage of laser beam welding risks thermally damaging the hermetic seal or seals formed beforehand.

There thus does not exist, in the prior art, a completely satisfactory solution—that is to say, a simple and reliable solution—for obtaining devices after encapsulation which are ultrathin, indeed even mechanically shapeable. In point of fact, such devices would make it possible to substantially improve the comfort of the patient and to envisage implantation in regions of the human body which are difficult for conventional devices to access. For example, an implantable device for cerebral stimulation provided in the form of an ultrathin and mechanically shapeable object might be implanted in the head, under the scalp, instead of being implanted conventionally in the top of the chest. Such an implantation at the closest to the region to be stimulated would make it possible to avoid recourse to long probes-electrodes connecting the chest to the head, such probes-electrodes exhibiting risks of breakage, in particular at the neck. For this type of implantation, the cerebral stimulation device would then have to exhibit a thickness of less than 1 mm and to be able to exhibit a radius of curvature of the order of a few centimeters.

The invention is targeted at overcoming the abovementioned disadvantages of the prior art. More specifically, the invention is targeted at providing a hermetic and, if necessary, biocompatible housing which can be easily produced in an ultrathin and, if appropriate, mechanically shapeable form. The invention is also targeted at providing a simple and reliable process for the encapsulation of a device and in particular of an implantable medical device, this process being in particular suited to the production of an ultrathin and/or shapeable assemblage. A device, housing or assemblage is regarded as “ultrathin” when it exhibits a maximum thickness of less than or equal to 3 mm and preferably of less than or equal to 1 mm, indeed even 500 μm.

Thus, a subject matter of the invention is a hermetic housing comprising:
a first element in the form of a sheet, made of at least one material chosen from a metal, a ceramic and a glass;
a second element having dimensions and a shape suitable for covering said first element, also made of at least one material chosen from a metal, a ceramic and a glass;
a first metal surround interposed between said first element and said second element, comprising an internal part positioned partially or completely inside the perimeter of said first element and an external part positioned completely outside said perimeter of said first element;
a first hermetic seal between said first element and said internal part of said first metal surround; and
a second hermetic seal between said second element and said external part of said first metal surround.

“Element in the form of a sheet” or simply “sheet” is understood to mean an element exhibiting a thickness of not greater than one tenth, preferably one fiftieth and more preferably one hundredth, of its smallest lateral dimension.

Advantageously, said first hermetic seal can be produced by a technique chosen from: soldering, solid state diffusion welding and, when said first element is at least partially made of ceramic, cosintering; and said second hermetic seal can be produced by welding.

According to a first embodiment of the invention, said second element can comprise:
an “upper” sheet, made of at least one material chosen from a metal, a ceramic and a glass;
a second metal surround interposed between said upper sheet and said first metal surround, comprising an internal part positioned partially or completely inside the perimeter of said upper sheet and an external part positioned completely outside said perimeter of said upper sheet; and
a third hermetic seal between said upper sheet and said internal part of said second metal surround;

said second hermetic seal being between the external parts of said first and second metal surrounds.

Advantageously, said third hermetic seal can be produced by a technique chosen from: soldering, solid state diffusion welding and, when said upper sheet is at least partially made of ceramic, cosintering.

According to a second embodiment of the invention, said second element can comprise an “upper” sheet made of metal, directly attached to the external part of said first metal surround via said second hermetic seal.

Both in the first and in the second embodiment of the invention, the external part of said or of each metal surround can exhibit, with respect to the corresponding internal part, an excess thickness over its face opposite said second hermetic seal. This facilitates the production of the second hermetic seal by welding, without, however, increasing the total thickness of the housing or compromising its flexibility, when mechanical shapeability is desired.

The hermetic housing can exhibit a thickness of less than or equal to 3 mm, preferably of less than or equal to 1 mm and more preferably of less than or equal to 500 μm, and can thus be “ultrathin”.

The housing can comprise at least one component, in particular electronic or electric component, mounted on said first element in the form of a sheet, directly or via a dielectric layer, and contained in a space delimited by said first element, said second element and said metal surround or surrounds. Advantageously, said or each component can be covered with a layer made of polymer material. In order to make possible the interconnection of the electronic component or components contained in the housing with external elements without compromising the leaktightness of the assembly, the housing can comprise at least one conductive track arranged on said first element in the form of a sheet, said or each conductive track extending over both faces and over the side face of said first element and being covered with an insulating material, except at its ends, forming lands.

Such a hermetic housing can be partially or completely coated with a layer made of polymer material.

In the case of a product which can be implanted in a human or animal body, the housing should be made completely or at least partially (in particular as regards its external surface) of biocompatible material or materials.

Other characteristics of the invention are presented in the description which follows the definition of the appended drawings. These characteristics relate in particular to the flexibility of the housing, the connection of hermetic electrodes/metal tracks or also the shaping of the lower or upper sheets, in particular in order to define a cavity in the housing.

Another subject matter of the invention is an implantable medical device comprising a hermetic housing as described above.

Yet another subject matter of the invention is a process for the encapsulation of a device (in particular of an implantable device, more particularly still of an implantable medical device), comprising the stages consisting in:
mounting at least one component of said device to be encapsulated on a first element in the form of a sheet, made of at least one material chosen from a metal, a ceramic and a glass;
superimposing, on said first element in the form of a sheet, a first metal surround comprising an internal part positioned partially or completely inside the perimeter of said first element and an external part positioned completely outside said perimeter of said first element;
forming a first hermetic seal between said first element and said internal part of said first metal surround;
superimposing, on said first metal surround, a second element with dimensions and a shape suitable for covering said first element, also made of at least one material chosen from a metal, a ceramic and a glass; and
forming a second hermetic seal between said second element and said external part of said first metal surround.

Advantageously, the stage of formation of said first hermetic seal can be carried out by a technique chosen from: soldering, solid state diffusion welding and, when said second element is at least partially made of ceramic, cosintering; and the stage of formation of said second hermetic seal can be carried out by welding.

According to a first embodiment of this process, said second element can comprise:
an “upper” sheet, made of at least one material chosen from a metal, a ceramic and a glass;
a second metal surround interposed between said upper sheet and said first metal surround and comprising an internal part positioned partially or completely inside the perimeter of said upper sheet and an external part positioned completely outside said perimeter of said upper sheet;

the process also comprising a stage of formation of a third hermetic seal between said upper sheet and said internal part of said second metal surround, said stage being carried out before the formation of said second hermetic seal between the external parts of said first and second metal surrounds.

Advantageously, the stage of formation of said third hermetic seal can be carried out by a technique chosen from: soldering, solid state diffusion welding and, when said upper sheet is at least partially made of ceramic, cosintering.

According to a second embodiment of this process, said second element can comprise an “upper” sheet made of metal, said second hermetic seal then being formed directly between said upper sheet and the external part of said first metal surround.

Other characteristics, details and advantages of the invention will emerge on reading the description, made with reference to the appended drawings given by way of example and in which:

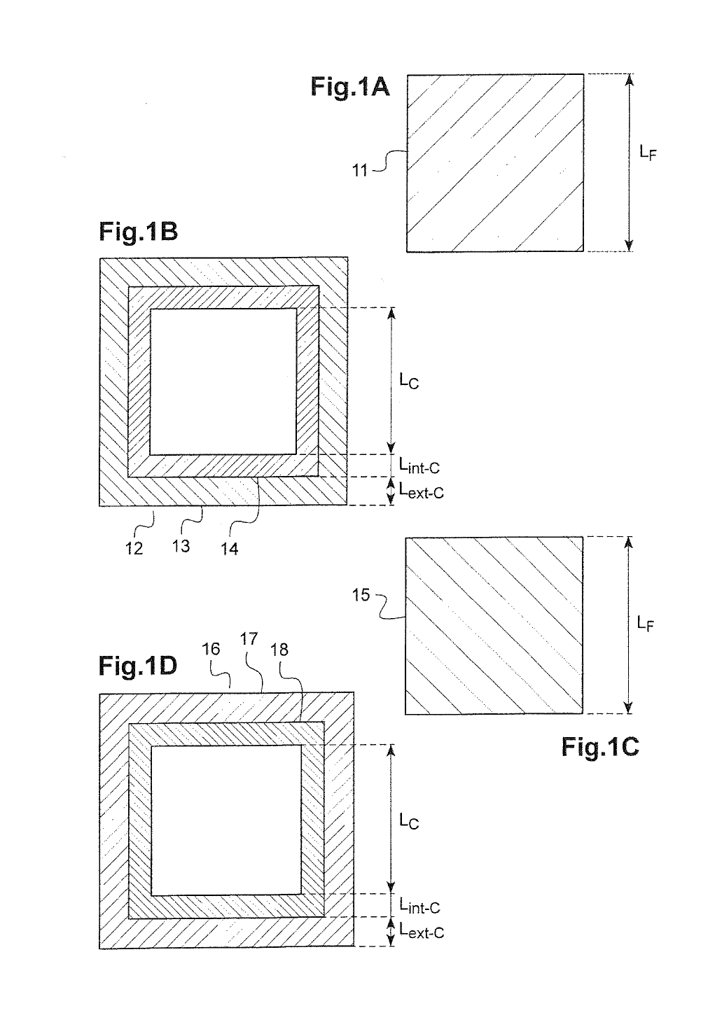

FIG. 1 (1A-1D) diagrammatically represents the different parts of a housing according to the first embodiment of the invention before they are assembled;

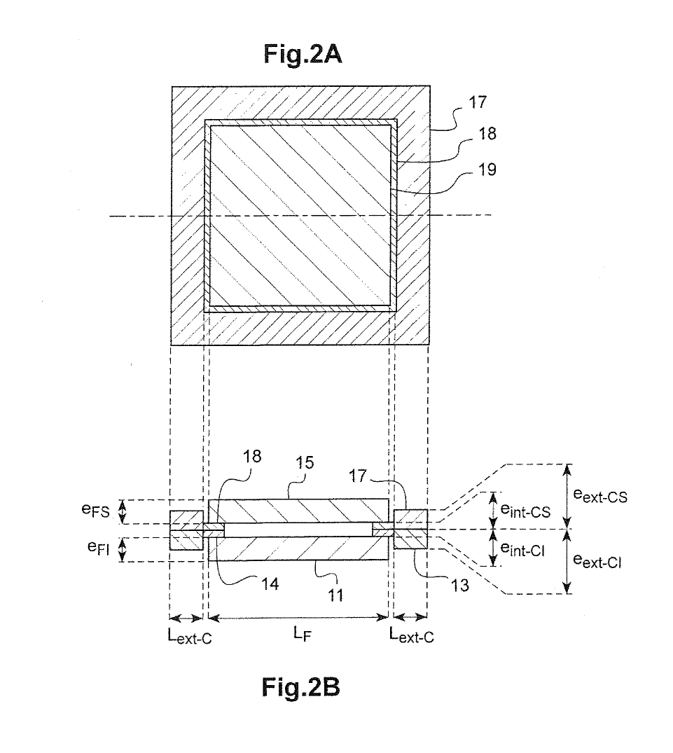

FIG. 2 represents a housing according to the first embodiment of the invention once assembled; in particular, FIG. 2A corresponds to a top view and FIG. 2B to a cross-sectional view;

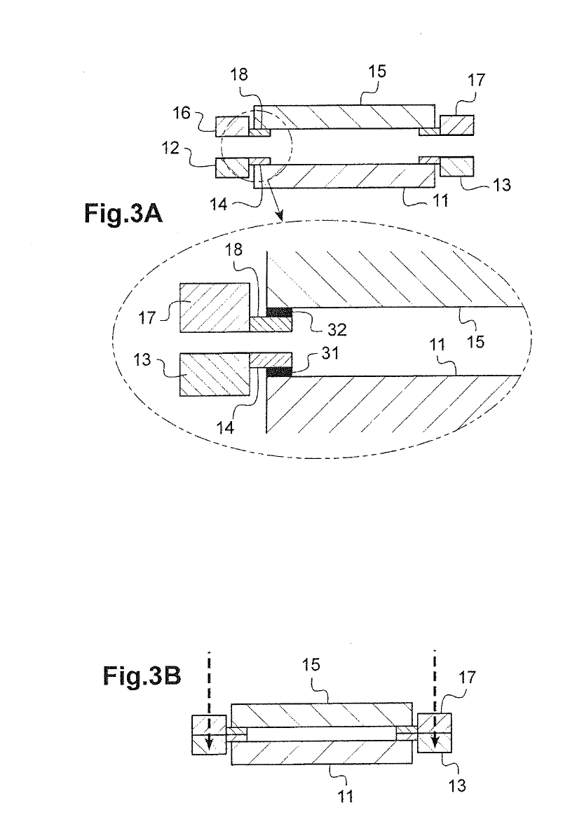

FIG. 3 (3A, 3B) illustrates the preparation by laser welding of the second hermetic seal of a housing according to the first embodiment of the invention;

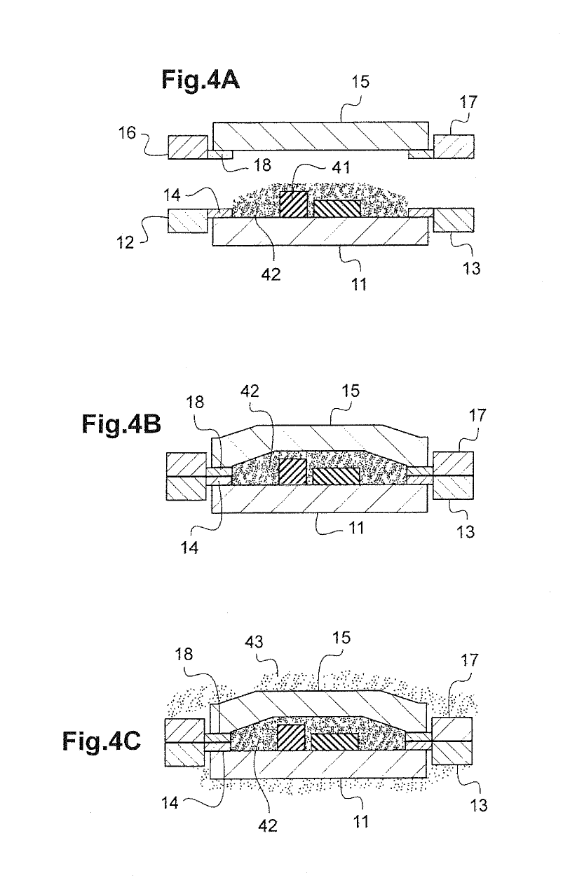

FIG. 4 illustrates the “pre-encapsulation” of electronic components inside a housing according to the first embodiment of the invention (4A, 4B), and the “post-encapsulation” of the housing itself (4C);

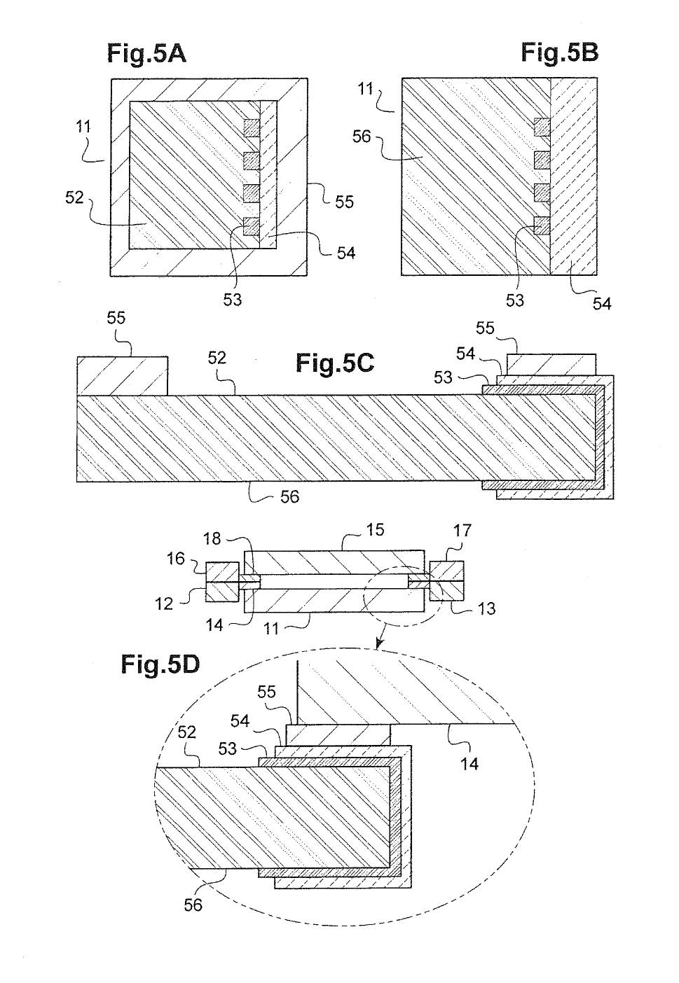

FIG. 5 (5A-5D) illustrates the preparation of a hermetic delivery for one or more conductive tracks;

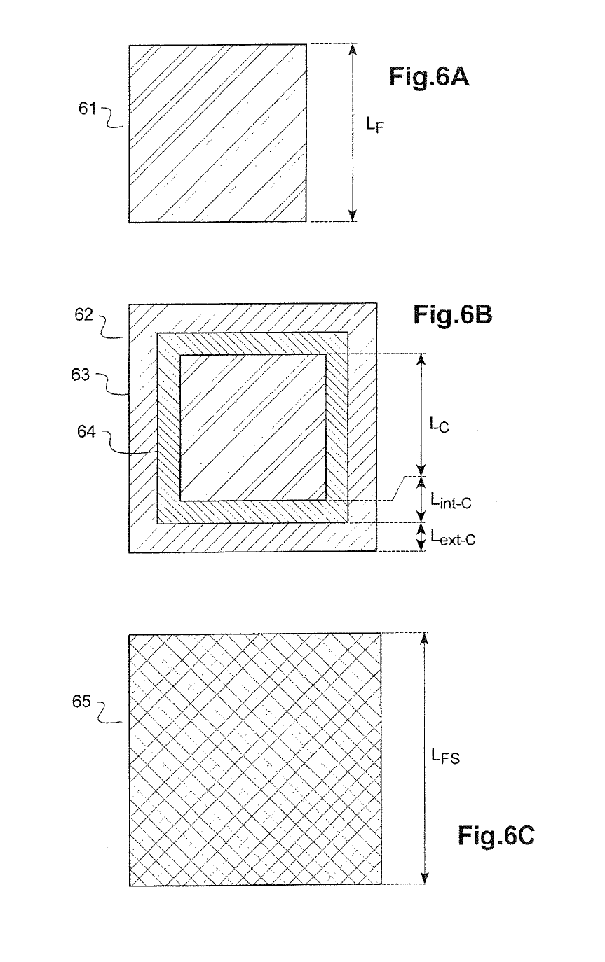

FIG. 6 (6A-6C) diagrammatically represents the different parts of a housing according to the second embodiment of the invention before they are assembled;

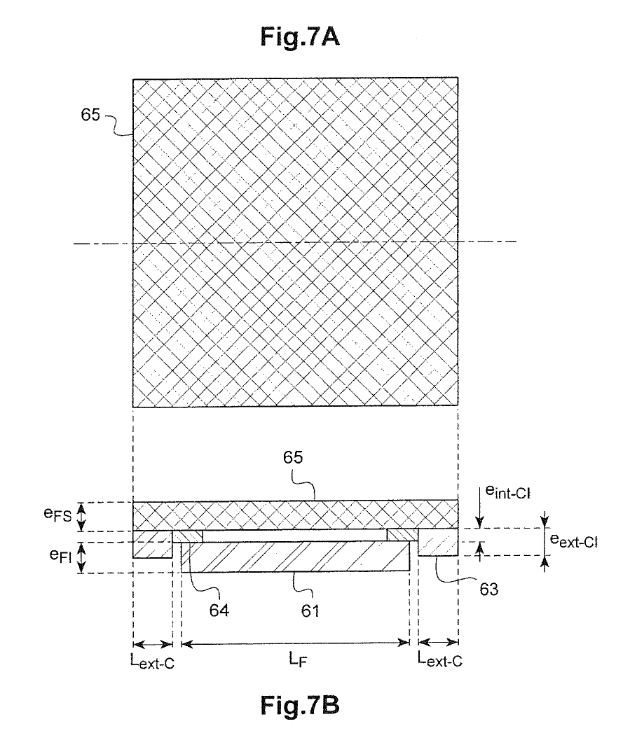

FIG. 7 represents a housing according to the second embodiment of the invention once assembled; in particular, FIG. 7A corresponds to a top view and FIG. 7B to a cross-sectional view;

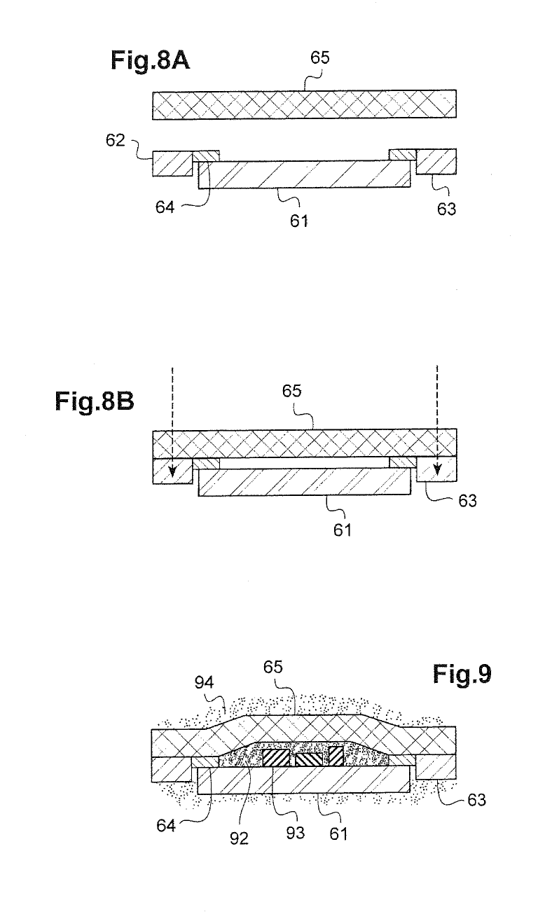

FIG. 8 (8A, 8B) illustrates the preparation by laser welding of the second hermetic seal of a housing according to the second embodiment of the invention;

FIG. 9 illustrates the “pre-encapsulation” of electronic components inside a housing according to the second embodiment of the invention, and the “post-encapsulation” of the housing itself; and

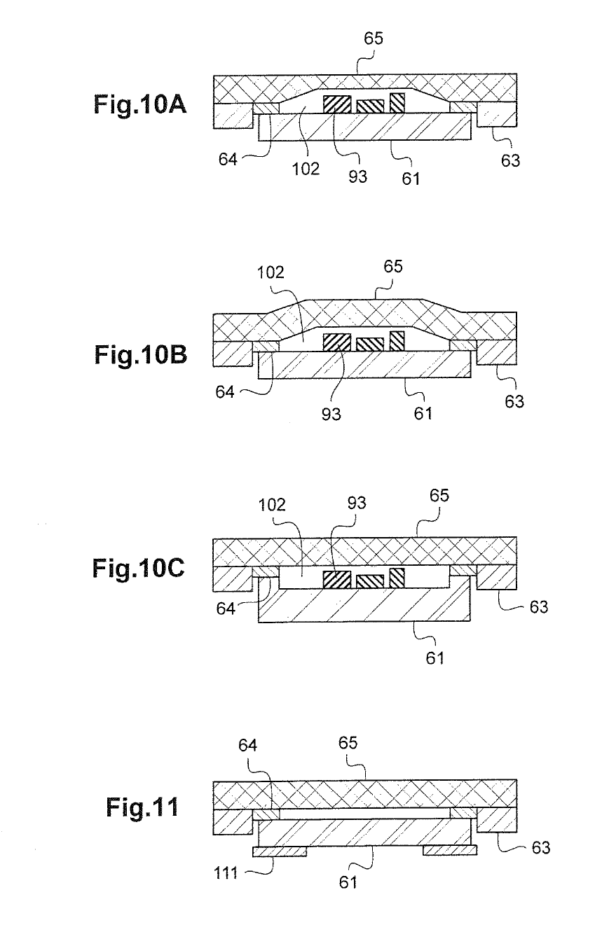

FIGS. 10 (10A, 10B, 10C) and 11 represent two alternative forms of housings according to the second embodiment of the invention;

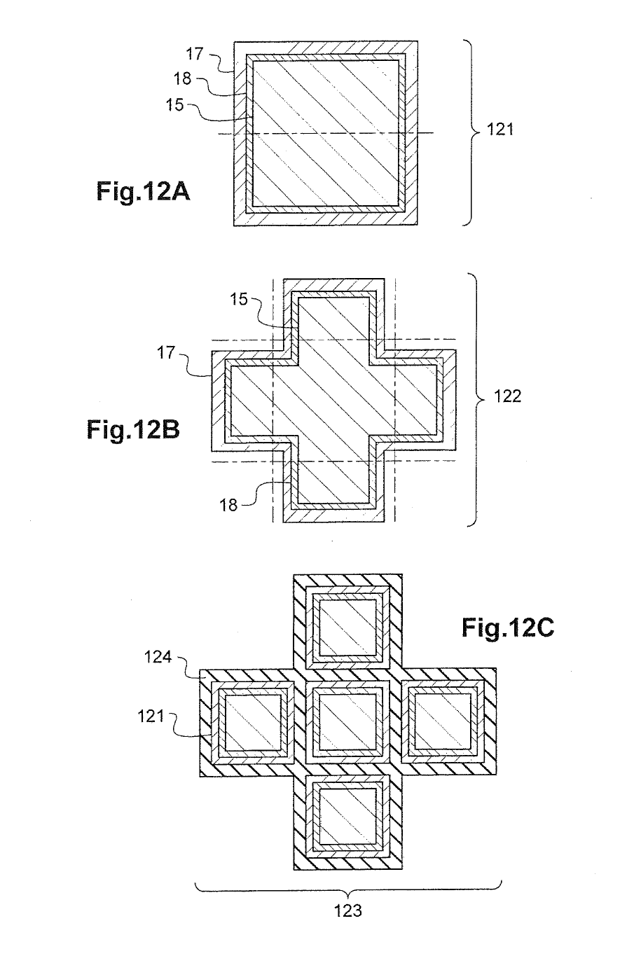

FIG. 12 (12A, 12B, 12C) illustrates means for improving the mechanical flexibility of a housing or of an assembly of housings according to the invention along several axes (FIG. 12A=reference);

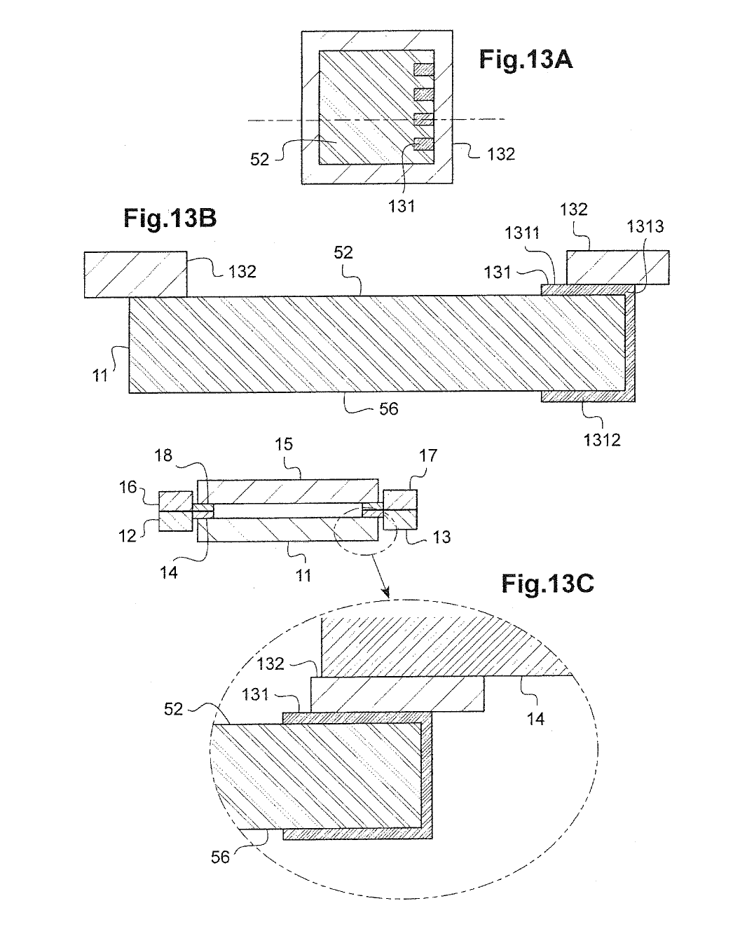

FIG. 13 (13A, 13B, 13C) illustrates the delivery of one or more electrical tracks forming electrodes or lands of a housing in accordance with the invention; the lower sheet of said housing is made of ceramic;

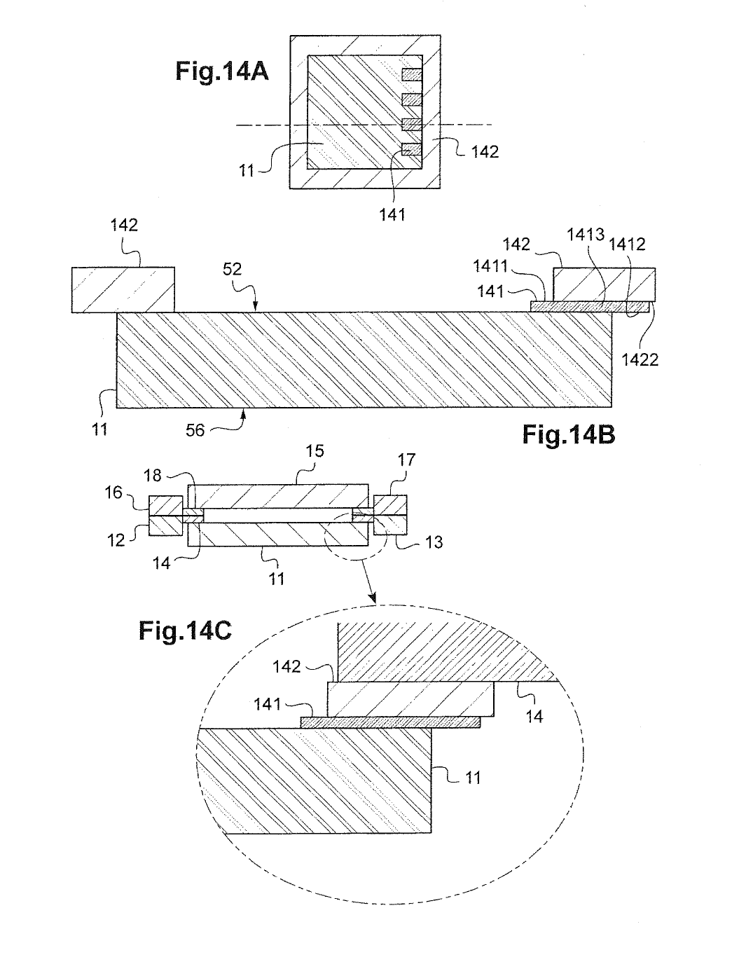

FIG. 14 (14A, 14B, 14C) illustrates another type of delivery of one or more electrical tracks forming electrodes or lands of a housing in accordance with the invention, in the case where the lower sheet of said housing is made of ceramic;

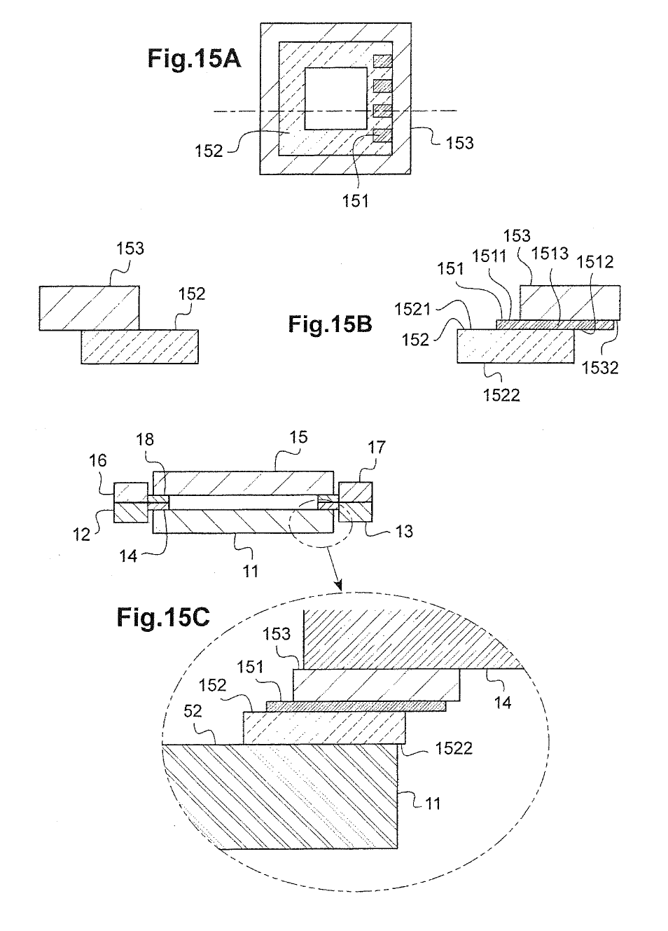

FIG. 15 (15A, 15B, 15C) illustrates yet another type of delivery of one or more electrical tracks forming electrodes or lands of a housing in accordance with the invention, in the case where the lower sheet of said housing is made of metal.

FIGS. 1 to 5 illustrate a housing according to a first embodiment of the invention and the various stages of its process of manufacture. In particular, FIG. 1 (top view) diagrammatically represents the various parts of the housing, before they are assembled. These parts are a first element in the form of a sheet, or “lower sheet” 11, acting as support for the device being encapsulated, a first metal surround 12, or “lower metal surround”, and a second element acting as cap, in its turn composed of a second element, also in the form of a sheet and referred to as “upper sheet” 15, and of a second metal surround, or “upper metal surround” 16.

FIG. 1A represents the lower sheet 11 which, in the exemplary embodiment considered here, is a square-shaped part, with a side denoted L_{F}, advantageously between 5 mm and 10 cm. Other shapes can be envisaged, in particular a circular shape. The thickness of the lower sheet 11, denoted e_{FI}, is advantageously less than 500 μm, preferably less than 50 μm. The lower sheet 11 is advantageously a part made of ceramic (preferably made of zirconia ZrO₂ stabilized with yttrium oxide Y₂O₃ at 3 mol % or more, or made of zirconia ZrO₂ stabilized with cerium oxide CeO₂) or made of metal (preferably made of titanium or made of titanium alloy). The lower sheet 11 can also be a part made of glass. One advantage of the ceramic or of the glass in comparison with the metal is its better transparency to electromagnetic or light radiation, which can be useful in some applications. One advantage of the stabilized zirconia in comparison with the glass is the better mechanical strength (in particular the better toughness). One advantage of the metal in comparison with the ceramic and with the glass is its better mechanical strength.

FIG. 1B represents the first metal surround, or lower metal surround, 12 which is a square-shaped part (more generally, a part matching the shape of the lower sheet) and which exhibits a central square-shaped opening with a side denoted L_{C}, advantageously between 5 mm and 10 cm. The lower metal surround 12 comprises two parts:
an external part 13, with a width denoted L_{ext-C} (advantageously of between 500 μm and 1 cm) and with a thickness denoted e_{ext-CI} (advantageously less than 500 μm, preferably less than 50 μm);
an internal part 14, with a width denoted L_{int-C} (advantageously of between 500 μm and 1 cm), and with a thickness denoted e_{int-CI} which is less than or equal to e_{ext-CI}.

The external part 13 and the internal part 14 constitute, in the case considered here, one and only one monolithic part 12. The lower metal surround 12 is advantageously a part made of titanium or made of titanium alloy.

FIG. 1C represents the upper sheet 15 which, in the exemplary embodiment considered here, is a square-shaped part, with a side equal to L_{F}. The thickness of the upper sheet 15, denoted e_{FS}, is advantageously less than 500 μm, preferably less than 50 μm. The upper sheet 15 is advantageously a part made of ceramic (preferably made of zirconia ZrO₂ stabilized with yttrium oxide Y₂O₃ at 3 mol % or more, or made of zirconia ZrO₂ stabilized with cerium oxide CeO₂) or made of metal (preferably made of titanium or made of titanium alloy). The upper sheet 15 can also be a part made of glass.

FIG. 1D represents the second metal surround, or upper metal surround, 16 which, in the exemplary embodiment considered here, is a square-shaped part (more generally, a part matching the shape of the lower sheet) exhibiting a central square-shaped opening with a side equal to L_{C}. The lower metal surround 16 itself also comprises two parts:
an external part 17 with a width equal to L_{ext-C} and with a thickness denoted e_{ext-CS} (advantageously less than 5 μm, preferably less than 50 μm);
an internal part 18 with a width equal to L_{int-C} and with a thickness denoted e_{int-CS} which is less than or equal to e_{ext-CS}.

The external part 17 and the internal part 18 constitute, in the case considered here, one and only one monolithic part 16. The upper metal surround 16 is advantageously a part made of titanium or made of titanium alloy.

FIG. 2 diagrammatically represents the housing once the various parts have been assembled (lower sheet 11, lower metal surround 12, upper sheet 15, upper metal surround 16). FIG. 2A is a top view, whereas FIG. 2B is a cross-sectional view along the axis represented as a dotted line in FIG. 2A. The external part 13 of the lower metal surround 12 is positioned completely outside the perimeter delimited by the lower sheet 11 and the internal part 14 of the lower metal surround 12 is positioned partially inside the perimeter delimited by the lower sheet 11. Likewise, the external part 17 of the upper metal surround 16 is positioned completely outside the perimeter delimited by the upper sheet 15 and the internal part 18 of the upper metal surround 16 is positioned partially inside the perimeter delimited by the upper sheet 15.

A first hermetic seal is formed between the lower sheet 11 and the internal part 14 of the lower metal surround 12. Advantageously, this hermetic seal is formed by soldering. In the case where the lower sheet 11 is made of zirconia and the lower metal surround 12 is made of titanium or made of titanium alloy, this soldering can be carried out in a furnace under high vacuum, at a temperature of the order of 1000° C., via a solder seal based on a titanium-nickel alloy. It will be possible, for example, to use a solder seal of the TiNi-50 type (50% Ni by weight) sold by Wesgo. Alternatively, it will be possible to use a solder seal comprising a larger amount of nickel (that is to say, greater than 50% by weight), the remainder of titanium being directly contributed by the titanium present in the lower metal surround 12. The solder seal can be positioned slightly set back (a few mm) from the edge of the lower sheet 11, in order in particular to prevent runoffs from the seal towards the outside of the perimeter delimited by the sheet 11 during the solder annealing. Before carrying out the soldering, a thin metal layer (titanium, gold, platinum, and the like) can be deposited beforehand (by physical vapor deposition, chemical vapor deposition, plating, coating, printing, and the like) on the edges of the lower sheet 11. As known in the prior art (see, for example, Jiang et al., “Technology advances and challenges in hermetic packaging for implantable medical devices”, Implantable Neural Prostheses 2, Biological and Medical Physics, Biomedical Engineering 2010, pp. 27-61), such a thin metal layer can facilitate the formation of the soldering of a metal part to a part made of ceramic. Before carrying out the soldering, a thin layer of oxide (alumina, silica, and the like) can be deposited beforehand (by physical vapor deposition, chemical vapor deposition, coating, printing, and the like) on the edges or all of the lower sheet 11, in order to limit the darkening of the part made of ceramic during the soldering (this possible darkening is due to the diffusion of the oxygen from the part made of ceramic toward the solder seal, resulting in a part made of ceramic which is substoichiometric in oxygen).

In an alternative form, the first hermetic seal can be produced by solid state diffusion soldering. In an alternative form, when the lower sheet is made of ceramic, the first hermetic seal can be produced by cosintering said lower sheet and said lower metal surround.

Another hermetic seal, known as “third hermetic seal”, is also formed between the upper sheet 15 and the internal part 18 of the upper metal surround 16. Advantageously, this hermetic seal is formed by soldering, by a similar process to that used to form the first hermetic seal between the lower sheet 11 and the internal part 14 of the lower metal surround 12.

Yet another hermetic seal, referred to as second hermetic seal, is formed between the external part 13 of the lower metal surround 12 and the external part 17 of the upper metal surround 16. Advantageously, this hermetic seal is formed by laser beam welding. This process is characterized by a localized contribution of heat, it being possible for the typical diameter of the laser beam to be of the order of a few hundred microns. Other welding processes with localized contribution of heat can be used, such as electron beam welding or resistance welding (Joule effect).

The fact that the external part 13 of the lower metal surround 12 is positioned completely outside the perimeter delimited by the lower sheet 11 and that the external part 17 of the upper metal surround 16 is positioned completely outside the perimeter delimited by the upper sheet 15 is particularly advantageous. This is because such a geometric configuration makes it possible to form a hermetic seal between the external part 13 of the lower metal surround 12 and the external part 17 of the upper metal surround 16, without, however, thermally damaging the hermetic seal between the lower sheet 11 and the internal part 14 of the lower metal surround 12 (this seal being positioned inside the perimeter delimited by the lower sheet 11) and/or the hermetic seal between the upper sheet 15 and the internal part 18 of the upper metal surround 16 (this seal being positioned inside the perimeter delimited by the upper sheet 15).

The fact that the thickness e_{ext-CI} (respectively e_{ext-CS}) of the external part 13 (respectively 17) of the lower metal surround 12 (respectively of the upper metal surround 16) is greater than or equal to the thickness e_{int-CI} (respectively e_{int-CS}) of the internal part 14 (respectively 18) makes it possible to contribute an amount of material sufficient to provide for the formation of a hermetic seal between the external part 13 and the external part 17, by a process such as laser beam welding. More particularly, the external part of each surround can exhibit an excess thickness oriented solely on the side opposite the second hermetic seal formed between the two surrounds. This makes it possible not to increase the total thickness of the assemblage.

The fact that the thickness e_{int-CI} (respectively e_{int-CS}) of the internal part 14 (respectively 18) of the lower metal surround 12 (respectively of the upper metal surround 16) is less than or equal to the thickness e_{ext-CI} (respectively e_{ext-CS}) of the external part 13 (respectively 17) makes it possible to provide for the thinness and the mechanical flexibility of the housing, this being the case even at the potentially thickest regions, that is to say the regions where a sheet/surround/surround/sheet stack occurs.

FIG. 3 (cross-sectional view) diagrammatically represents the laser beam welding process for the formation of the hermetic seal between the external part 13 of the lower metal surround 12 and the external part 17 of the upper metal surround 16.

FIG. 3A represents the lower part of the housing (which comprises the lower sheet 11 and the lower metal surround 12) and the upper part of the housing (which comprises the upper sheet 15 and the upper metal surround 16), after the formation of the hermetic seals between the sheets and the metal surrounds but before the formation of the second hermetic seal between the two metal surrounds. In the case represented in FIG. 3A, the hermetic seal between the lower sheet 11 and the internal part 14 of the lower metal surround 12 was formed by soldering, via a solder seal 31; likewise, the hermetic seal between the upper sheet 15 and the internal part 18 of the upper metal surround 16 was formed by soldering, via a solder seal 32.

Advantageously, elements—components, or devices (and in particular electronic components)—to be encapsulated in a hermetic housing according to the invention are positioned between the lower sheet 11 and the upper sheet 15 only after the formation of the hermetic seal between the sheets and the metal surrounds. This is because the process for the formation of the hermetic seal between the sheets and the metal surrounds generally involves a stage in a furnace at high temperature, which is liable to damage the elements to be encapsulated.

FIG. 3B represents the formation of said second hermetic seal between the two metal surrounds by laser beam welding. The hermetic seal is formed between the external part 13 of the lower metal surround 12 and the external part 17 of the upper metal surround 16, by localized contribution of heat in this region. The laser beam is represented diagrammatically by two arrows as a dotted line, entering the external part 17 of the upper metal surround 16 and the external part 13 of the lower metal surround 12. Given that the external parts of the metal surrounds 12 and 16 are positioned completely outside the perimeter delimited by the sheets 11 and 15, the welding process does not thermally damage either the hermetic seals formed beforehand between the sheets and the metal surrounds (in particular the solder seals 31 and 32) or the elements to be encapsulated positioned beforehand between the two sheets.

The elements to be encapsulated can form the subject of a “pre-encapsulation”, that is to say of a coating by a polymer material prior to the formation of the second hermetic seal.

FIG. 4A represents the lower part and the upper part of the housing after the formation of the hermetic seal between the sheets and the metal surrounds but before the formation of the hermetic seal between the two metal surrounds. Elements 41 to be encapsulated are attached to the lower sheet 11, optionally via a dielectric layer if said lower sheet is metallic. In particular, the lower sheet 11 can constitute a substrate on which components 41 are assembled by techniques of flip chip or wire bonding type. The components 41 can be integrated circuits (preferably on a thinned silicon substrate), discrete passive components (in particular capacitors or coils), batteries made of thin layers (on a thinned silicon substrate or on an ultrathin sheet made of polymer, ceramic, metal or glass), photovoltaic modules made of thin layers (on an ultrathin sheet made of polymer, ceramic, metal or glass), photovoltaic modules made of crystalline silicon (preferably on a thinned silicon substrate), light-emitting diodes (on a thinned substrate) or any other electronic, photonic, energy recovery or energy storage component.

The components 41 are covered with a “pre-encapsulation” layer made of polymer 42. The role of the pre-encapsulation layer 42 is to mechanically protect the components 41. Advantageously, the maximum thickness of the preencapsualtion layer 42 is less than 300 μm. Advantageously and as represented in FIG. 4A, the thickness of the pre-encapsulation layer 42 in the regions located close to the internal part 14 of the lower metal surround 12 is less than or approximately equal to the sum e_{int-CI}+e_{int-CS}. Advantageously, the slope of the relief of the pre-encapsulation layer 42 is low. More specifically, the slope of the relief of the pre-encapsulation layer 42 is everywhere less than 20%. The pre-encapsulation layer 42 is, for example, based on silicones or polyurethanes. These polymers exhibit the advantage of being flexible, easily shaped by molding processes and commonly used in medical implants. The pre-encapsulation layer 42 can be formed by a molding, deposition or rolling process.

FIG. 4B represents the housing after the formation of the hermetic seal between the two metal surrounds. Advantageously, the upper sheet 15 is slightly distorted in order to match in compliable fashion the reliefs of the pre-encapsulation layer 42. This is rendered possible because the slope of the relief of the pre-encapsulation layer 42 is low and because the upper sheet 15 is ultrathin. Such a configuration greatly restricts the risk of damage to the upper sheet 15 during and after the process of formation of the hermetic seal between the two metal surrounds.

Advantageously, the “free surface” of the upper sheet 15 (that is to say, the central surface of the upper sheet 15 which does not form a hermetic seal with the internal part 18 of the upper metal surround 16) is slightly greater than the “free surface” of the lower sheet 11 (that is to say, the central surface of the lower sheet 11 which does not form a hermetic seal with the internal part 14 of the lower metal surround 12). This makes it possible for the upper sheet 15 to slightly distort in order to match in a compliable fashion the reliefs of the pre-encapsulation layer 42.

The encapsulated implantable medical device diagrammatically represented in FIG. 4B exists in the form of an ultrathin and mechanically shapeable object. The thickness of the object:
at the stack 13/17 is preferably less than 100 μm (if the thickness of each of the external parts of the metal surrounds is less than 50 μm) and can be of the order of 60 μm (if the thickness of each of the external parts of the metal surrounds is of the order of 30 μm),
at the stack 11/14/18/15 is preferably less than 150 μm (if the thickness of each of the sheets is less than 50 μm and if the thickness of each of the internal parts of the metal surrounds is less than 25 μm) and can be of the order of 100 μm (if the thickness of each of the sheets is of the order of 40 μm and if the thickness of each of the internal parts of the metal surrounds is of the order of 10 μm),
at the stack 11/42/15 is preferably less than 400 μm (if the thickness of each of the sheets is less than 50 μm and if the maximum thickness of the pre-encapsulation layer is less than 300 μm) and can be of the order of 250 μm (if the thickness of each of the sheets is of the order of 40 μm and if the maximum thickness of the pre-encapsulation layer is of the order of 170 μm).

Thus, the thickness of the object at its thickest region (that is to say, at the stack 11/42/15) can be of the order of 250 μm. In terms of mechanical flexibility, the flexibility of the object is limited by the sheets (which are parts made of ceramic, metal or glass) and the metal surrounds. The flexibility is not limited by the pre-encapsulation layer 42 as it is a layer made of polymer which remains flexible even for high thicknesses. The flexibility will thus be limited essentially by the thickness of the stack 11/14/18/15, which can be of the order of 100 μm, as described in detail above. Such a thickness is sufficiently low to provide the final object with good mechanical flexibility.

The housing can also be coated with a polymer material, after the formation of the second hermetic seal (“post-encapsulation”).

FIG. 4C represents a layer made of polymer 43, referred to as post-encapsulation layer, which coats the lower sheet 11, the upper sheet 15 and the hermetic seals formed laterally between these two sheets. A first role of the post-encapsulation layer 43 is to mechanically protect the assembly of the device and of the housing, in particular the lower sheet 11 and the upper sheet 15. A second role of the post-encapsulation layer 43 is to obtain a final device, the edges of which are not sharp. Advantageously, the maximum thickness of the post-encapsulation layer 43 is less than 300 μm. The post-encapsulation layer 43 is, for example, based on silicones or polyurethanes. The post-encapsulation layer 43 can be formed by a molding, deposition or rolling process.

Electronic devices or components encapsulated inside a housing according to the invention generally have to be able to communicate with the outside by means of electrodes. For example, a neural stimulator or pacemaker has to be able to deliver pulses to tissues, directly or via probes. It is therefore necessary to make possible the delivery of one or more electrical tracks forming electrodes or lands, this being done without compromising the hermiticity of the housing. A possible solution to this problem is illustrated by FIG. 5.

FIG. 5A (top view), FIG. 5B (bottom view) and FIG. 5C (cross-sectional view along the axis represented as a dotted line in FIG. 5A) represent the delivery of electrodes 53 at the lower sheet 11. As mentioned above, the lower sheet 11 is advantageously a part made of ceramic, of metal or of glass. In the case where the lower sheet 11 is made of metal, it has to be covered, over its lower face and its upper face and also over its side faces, with a thin electrically insulating layer, for example a thin layer of alumina deposited by chemical vapor deposition. Electrically conductive tracks 53 are formed on one of the edges of the lower sheet 11, so that the conductive tracks 53 connect the upper face 52 and the lower face 56 of the lower sheet 11 via the side face of the lower sheet 11. The width of the conductive tracks 53 is advantageously between 10 μm and 1 mm. The thickness of the conductive tracks 53 is advantageously between 10 nm and 1 μm. The conductive tracks 53 are, for example, thin metal layers (gold, platinum, and the like) deposited by physical vapor deposition, chemical vapor deposition, plating, coating or printing. The deposition stage can be combined with a stage of structuring, for example by photolithography, chemical etching, plasma etching, mechanical etching or laser etching. The conductive tracks 53 are covered in a compliable fashion with a thin electrically insulating layer 54. Only the ends of the conductive tracks 53 (at the upper face 52 and the lower face 56 of the lower sheet 11) are not covered with the thin insulating layer 54. The thickness of the thin insulating layer 54 is advantageously between 10 nm and 1 μm. The thin insulating layer 54 is, for example, a thin layer of silica, silicon nitride, alumina or zirconia deposited by physical vapor deposition, chemical vapor deposition, coating or printing. The deposition stage can be combined with a structuring stage. An electrically insulating surround 55 is formed on the edges of the upper face 52 of the lower sheet 11; it has the role of rendering said edges flat, in order for the internal part 14 of the lower metal surround to be able to be positioned on a flat surface. The insulating surround 55 partially covers the thin insulating layer 54 and the conductive tracks 53. The thickness of the insulating surround 55 is advantageously between 100 nm and 50 μm. The insulating surround 55 can, for example, be a layer of silica, silicon nitride, alumina or zirconia deposited by physical vapor deposition, chemical vapor deposition, coating or printing. The deposition stage can be combined with a structuring stage. An insulating material which acts as diffusion barrier, such as a dense ceramic, is particularly advantageous, in order to prevent the diffusion of metal entities through the insulating surround 55 during the formation of the hermetic seal between the lower sheet 11 and the internal part 14 of the lower metal surround 12. Alternatively, the insulating surround 55 can be a part made of ceramic (preferably made of zirconia ZrO₂ stabilized with yttrium oxide Y₂O₃ at 3 mol % or more, or made of zirconia ZrO₂ stabilized with cerium oxide CeO₂), assembled by cosintering with the lower sheet 11.

FIG. 5D (cross-sectional view) diagrammatically represents the housing once the various parts have been assembled (lower sheet 11 with the delivery of electrodes 53, lower metal surround 12, upper sheet 15 and upper metal surround 16).

FIGS. 6 to 8 illustrate a housing according to a second embodiment of the invention and the various stages of its process of manufacture. This second embodiment differs from the first embodiment described above in that the second element acting as cap is composed of the second sheet (or upper sheet) alone, which is made of metal, the second surround (or upper surround) being absent.

FIG. 6 (top view) diagrammatically represents the various parts of such a housing, before they are assembled.

FIG. 6A represents the lower sheet 61. In the case represented in FIG. 6A, the lower sheet 61 is a square-shaped part, with a side denoted L_{F}, advantageously of between 5 mm and 10 cm. Other shapes can be envisaged, in particular a circular shape. The thickness of the lower sheet 61, denoted e_{FI}, is advantageously of less than 500 μm, preferably of less than 50 μm. The lower sheet 61 is advantageously a part made of ceramic (preferably made of zirconia ZrO₂ stabilized with yttrium oxide Y₂O₃ at 3 mol % or more, or made of zirconia ZrO₂ stabilized with cerium oxide CeO₂) or made of metal (preferably made of titanium or made of titanium alloy). The lower sheet 61 can also be a part made of glass.

FIG. 6B represents the sole metal surround of the housing which, for the sake of terminological coherence with the first embodiment, will be known as “first metal surround” or “lower metal surround” 62. In the exemplary embodiment considered here, the lower metal surround 62 is a square-shaped part exhibiting a central square-shaped opening with a side, denoted L_{C}, advantageously of between 5 mm and 10 cm. As in the first embodiment of the invention, the lower metal surround 62 comprises two parts:
an external part 63 with a width denoted L_{ext-C} (advantageously of between 500 μm and 1 cm) and with a thickness denoted e_{ext-CI} (advantageously of less than 500 μm, preferably of less than 50 μm);
an internal part 64 with a width denoted L_{int-C} (advantageously of between 500 μm and 1 cm) and with a thickness denoted e_{int-CI} which is less than or equal to e_{ext-CI}.

In the exemplary embodiment considered here, the external part 63 and the internal part 64 constitute one and only one monolithic part 62. The lower metal surround 62 is advantageously a part made of titanium or made of titanium alloy.

FIG. 6C represents the upper metal sheet 65. In the case represented in FIG. 6C, the upper metal sheet 65 is a square-shaped part, with a side denoted L_{FS}, advantageously of between 5 mm and 10 cm. The side L_{FS} of the upper metal sheet 65 is greater than the side L_{F} of the lower sheet 61, in order to make possible the positioning of the external part 63 of the lower metal surround 62 at the vertical of the periphery of the upper metal sheet 65 and then the formation of a hermetic seal between the external part 63 of the lower metal surround 62 and the periphery of the upper metal sheet 65 (see below). The thickness of the upper metal sheet 65, denoted e_{FS}, is advantageously of less than 500 μm, preferably of less than 50 μm. The upper metal sheet 65 is advantageously a part made of titanium or made of titanium alloy.

FIG. 7 diagrammatically represents the housing of FIG. 6 once the various parts have been assembled (lower sheet 61, lower metal surround 62, upper metal sheet 65). FIG. 7A is a top view, whereas FIG. 7B is a cross-sectional view along the axis represented as a dotted line in FIG. 7A. The external part 63 of the lower metal surround 62 is positioned completely outside the perimeter delimited by the lower sheet 61, and the internal part 64 of the lower metal surround 62 is positioned partially inside the perimeter delimited by the lower sheet 61. A first hermetic seal is formed between the lower sheet 61 and the internal part 64 of the lower metal surround 62. Advantageously, this hermetic seal is formed by soldering. Alternatively, this hermetic seal can be formed by solid state diffusion welding or by cosintering. A second hermetic seal is formed between the external part 63 of the lower metal surround 62 and the periphery of the upper metal sheet 65. Advantageously, this hermetic seal is formed by laser beam welding. Other welding processes with localized contribution of heat can be used, such as electron beam welding or resistance welding (Joule effect).

As was explained concerning the first embodiment, it is advantageous for the external part 63 of the lower metal surround 62 to be positioned completely outside the perimeter delimited by the lower sheet 61 and for the thickness e_{ext-CI} of the external part 63 of the lower metal surround 62 to be greater than or equal to the thickness e_{int-CI} of the internal part 64.

FIG. 8 (cross-sectional view) diagrammatically represents the laser beam welding process for the formation of the hermetic seal between the external part 63 of the lower metal surround 62 and the periphery of the upper metal sheet 65.

FIG. 8A represents the lower part of the housing (which comprises the lower sheet 61 and the lower metal surround 62) and the upper part of the housing (which comprises the upper metal sheet 65) after the formation of the hermetic seal between the lower sheet 61 and the lower metal surround 62 but before the formation of the hermetic seal between the lower metal surround 62 and the upper metal sheet 65. As is explained concerning the first embodiment, it is advantageous for element—components, or devices (and in particular electronic components)—to be encapsulated in a hermetic housing according to the invention to be positioned between the lower sheet 61 and the upper sheet 65 only after the formation of the hermetic seal between the lower sheet 61 and the lower metal surround 62. This is because the formation of this seal generally involves a stage in a furnace at high temperature, which is liable to damage the elements to be encapsulated.

FIG. 8B represents the formation of the second hermetic seal between the lower metal surround 62 and the upper metal sheet 65 by laser beam welding. The hermetic seal is formed between the external part 63 of the lower metal surround 62 and the periphery of the upper metal sheet 65, by localized contribution of heat in this region. The laser beam is represented diagrammatically by two arrows as a dotted line which enters the periphery of the upper metal sheet 65 and the external part 63 of the lower metal surround 62. As in the case of the first embodiment, given that the external part 63 of the lower metal surround 62 is positioned completely outside the perimeter delimited by the lower sheet 61, the welding process does not thermally damage either the hermetic seal formed beforehand between the lower sheet 61 and the lower metal surround 62 or the elements to be encapsulated positioned beforehand between the two sheets.

In the same way as in the first embodiment, the encapsulation can be supplemented by a pre-encapsulation 92 which mechanically protects the elements 93 and/or a post-encapsulation 94, as represented in FIG. 9 (cross-sectional view).

In the same way as in the first embodiment, a hermetic delivery of one or more electrodes can be installed.

The invention allows various alternative forms and improvements.

For example, a dehydrating agent can be introduced into the internal space defined by the housing, in particular by deposition of a thin layer of a silica gel on the internal face of one of the sheets or by use of a pre-encapsulation polymer charged with silica particles (in particular nanoparticles).

The sheets can exhibit a controlled relief, in particular in order to define a cavity. For example, as represented diagrammatically in FIG. 10A in the case of the second embodiment, the upper metal sheet 65 can exhibit a concave shape. This makes it possible, after assembling the various parts of the housing, to define a cavity 102 comprising the components 93.

In another example, represented diagrammatically in FIG. 10B in the case of a second embodiment, the upper metal sheet 65, which was initially flat, has been shaped by stamping or hydroforming or any other mechanical process. As may be observed, the internal and external faces of the sheet 65 are both concave in shape. This makes it possible, after assembling the various parts of the housing, to define a cavity 102 comprising the components 93. The thickness of the object described in FIG. 10B in its thickest region (that is to say, at the stack 61/102/65) can, for example, be of the order of 3 mm (if the thickness of each of the sheets 61 and 65 is of the order of 500 μm and if the maximum height of the cavity 102 is of the order of 2 mm). At the other end of the thickness range advantageously considered in the present invention, the thickness of the object described in FIG. 10B in its thickest region (that is to say, at the stack 61/102/65) can be of the order of 250 μm (if the thickness of each of the sheets 61 and 65 is of the order of 40 μm and if the maximum height of the cavity 102 is of the order of 170 μm).

In another example, represented diagrammatically in FIG. 10C in the case of the second embodiment, the lower sheet 61 can exhibit a U shape. In the case where the lower sheet 61 is a part made of ceramic, such a U shape can be obtained by cosintering a flat sheet made of ceramic and a surround made of ceramic. The U shape of the lower sheet 61 makes it possible, after assembling the various parts of the housing, to define a cavity 102 comprising the components 93. In the case where the lower sheet 61 is a part made of ceramic, it is preferable to define the cavity 102 by giving a U shape to the lower sheet 61, rather than to simply increase the thickness of the internal part 64 of the lower metal surround 62; this is because the increase of the thickness of the internal part 64 of the lower metal surround 62 has the consequence of increasing the amount of metal in the housing, which can present problems in the case of implantable applications as regards compatibility of the housing with magnetic resonance imaging (MRI).

Finally, the upper metal sheet 65 can exhibit a concave shape, solely on its internal face or simultaneously on its internal and external faces, and/or the lower sheet 61 can exhibit a U shape.

The alternative forms of FIGS. 10A, 10B and 10C also relate to the first embodiment.

In the case of a sheet made of ceramic or of glass, a protective metal surround can be added to the external face, in order to reinforce the mechanical strength. For example, as represented in FIG. 11 in the case of the second embodiment, a protective metal surround 111 is attached to the lower face of the lower sheet 61. The central opening of the protective metal surround 111 can be larger or smaller according to the application targeted. This alternative form also relates to the first embodiment.

In the case of a sheet made of ceramic, a deposition of thin layers can be carried out on the external face, in order to strengthen the resistance to attacks by biological tissues or fluids and to limit the aging of the ceramic. This is because ceramics, such as zirconia stabilized with yttrium oxide, are liable to age in humid environments. The thin layers can, for example, be based on metals (titanium, gold, platinum, and the like), on oxides (alumina, silica, and the like) or on hydrophobic polymers (polytetrafluoroethylene, and the like).

FIG. 12A (top view) represents a housing 121 with a simple geometric shape. With such a geometric shape, the housing can be curved along an axis (for example, the axis represented as a dotted line in FIG. 12A), which makes it possible to position the housing in compliable fashion over objects of cylindrical type. However, it is more difficult to curve the housing 121 along several axes as this produces strong mechanical stresses liable to damage the housing. Thus, it is more difficult to position the housing 121 in compliable fashion over noncylindrical objects (for example objects of spherical type).

If a housing exhibits a more complex geometric shape, then it can be curved along one or more axes (in particular several axes which are nonparallel with one another) without generating strong mechanical stresses. The housing can, for example, exhibit a cross shape, thus several branches, with at least one potential axis of curvature at each branch. This then allows the housing to be positioned in compliable fashion over objects of varied shapes which are not necessarily cylindrical (for example objects of spherical type). By way of example, FIG. 12B (top view) represents a housing 122 having a cross shape with four branches. With such a geometric shape, the housing 122 can be curved along at least four axes (for example axes represented as dotted lines in FIG. 12B).

It is possible to manufacture an assemblage or device of several housings, this assemblage having a degree of shapeability or mechanical flexibility which is greater than that of a single housing (for a given shape and given dimensions). By way of example, FIG. 12C represents an assemblage 123 of several housings 121. The assemblage 123 has a shape and dimensions which are similar to those of the housing 122 represented in FIG. 12B. However, the assemblage 123 potentially has a greater degree of shapeability or mechanical flexibility than that of the housing 122. This is because the housings 121 included in the assemblage 123 are connected mechanically by a flexible common support 124, for example made of polymer. This polymer can be biocompatible, for example a polymer based on silicone or on polyurethane. The flexible common support 124 can, for example, be a sheet made of polymer to which the housings 121 are adhesively bonded (on one or both faces); the support 124 can also be an overmolded part made of polymer which partially or completely coats the housings 121. In addition to its mechanical role, the support 124 can act as electrical interconnection. Thus, the support 124 can include metal tracks or wires which make it possible to electrically interconnect the various housings 121; the support 124 can also include electrical connectors (which make it possible to connect the assemblage 123 to other components). Finally, the support 124 can include electronic components which it is not necessary to encapsulate in the housings 121 (for example, a coil or antenna, making it possible to receive or transmit information or energy).

FIGS. 13, 14 and 15 exhibit alternative arrangements for making possible the delivery of one or more electrical tracks forming electrodes or lands, this being done without compromising the hermeticity of the housing. FIGS. 13 and 14 describe the case where the lower sheet 11 is a part made of ceramic and FIG. 15 describes the case where the lower sheet 11 is a part made of metal.

FIG. 13A (top view) and FIG. 13B (cross-sectional view along the axis represented as a dotted line in FIG. 13A) represent the delivery of electrodes 131 at an assemblage including the lower sheet 11 and an additional surround 132. In the case considered here, the lower sheet 11 is a part made of ceramic, preferably made of zirconia ZrO₂ stabilized with yttrium oxide Y₂O₃ at 3 mol % or more or made of zirconia ZrO₂ stabilized with cerium oxide CeO₂. The additional surround 132 is also a part made of ceramic. The electrodes 131 are metal tracks, preferably made of platinum or made of platinum alloy. The metal tracks 131 form hermetic ceramic-metal feedthroughs between the lower sheet 11 and the surround 132. Each metal track 131 connects the upper face 52 and the lower face 56 of the lower sheet 11 via the side face of the lower sheet 11.

Each metal track 131 exhibits:
a portion 1313 sandwiched between the lower sheet 11 and the surround 132,
a first end 1311 accessible on the upper face 52 of the lower sheet 11,
a second end 1312 accessible on the lower face 56 of the lower sheet 11.

The assemblage, including the metal tracks 131, the lower sheet 11 and the additional surround made of ceramic 132, is preferably prepared by cosintering.

FIG. 13C (cross-sectional view) diagrammatically represents the housing once the various parts have been assembled. A first hermetic seal is formed between the additional surround 132 and the internal part 14 of the lower metal surround 12. Advantageously, this first seal is formed by soldering.

FIG. 14A (top view) and FIG. 14B (cross-sectional view along the axis represented as a dotted line in FIG. 14A) represent the delivery of electrodes 141 at an assemblage including the lower sheet 11 and an additional surround 142. In the case considered here, the lower sheet 11 is a part made of ceramic, preferably of zirconia ZrO₂ stabilized with yttrium oxide Y₂O₃ at 3 mol % or more or made of zirconia ZrO₂ stabilized with cerium oxide CeO₂. The additional surround 142 is also a part made of ceramic. The electrodes 141 are metal tracks, preferably made of platinum or made of platinum alloy. The metal tracks 141 form hermetic ceramic-metal feedthroughs between the lower sheet 11 and the surround 142.

Each metal track 141 exhibits:
a portion 1413 sandwiched between the lower sheet 11 and the surround 142,
a first end 1411 accessible on the upper face 52 of the lower sheet 11,
a second end 1412 accessible on the lower face 1422 of the additional surround 142.

The assemblage, including the metal tracks 141, the lower sheet 11 and the surround 142, is preferably prepared by cosintering.

FIG. 14C (cross-sectional view) diagrammatically represents the housing once the various parts have been assembled. A first hermetic seal is formed between the surround 142 and the internal part 14 of the lower metal surround 12. Advantageously, this first seal is formed by soldering.

FIG. 15A (top view) and FIG. 15B (cross-sectional view along the axis represented as a dotted line in FIG. 15A) represent the delivery of electrodes 151 at an assemblage including a first additional surround 152 and a second additional surround 153. The two surrounds 152, 153 are parts made of ceramic, preferably made of zirconia ZrO₂ stabilized with yttrium oxide Y₂O₃ at 3 mol % or more or made of zirconia ZrO₂ stabilized with cerium oxide CeO₂. The electrodes 151 are metal tracks, preferably made of platinum or made of platinum alloy. The metal tracks 151 form hermetic ceramic-metal feedthroughs between the surround 152 and the surround 153.

Each metal track 151 exhibits:
a portion 1513 sandwiched between the first additional surround 152 and the second additional surround 153,
a first end 1511 accessible on the upper face 1521 of the first additional surround 152,
a second end 1512 accessible on the lower face 1532 of the second additional surround 153.

The assemblage, including the metal tracks 151, the surround 152 and the surround 153, is preferably prepared by cosintering.

FIG. 15C (cross-sectional view) diagrammatically represents the housing once the various parts have been assembled. A first hermetic seal is formed between the second additional surround 153 and the internal part 14 of the lower metal surround 12. Advantageously, this first seal is formed by soldering. Another hermetic seal is also formed between the first additional surround 152 and the lower sheet 11. In the case considered here, the lower sheet 11 is a part made of metal. Advantageously, the seal between the first additional surround 152 and the lower sheet 11 is formed by soldering, simultaneously with the formation of the seal between the second additional surround 153 and the internal part 14 of the lower metal surround 12.

Another method for making possible the delivery of one or more electrical tracks forming electrodes or lands consists in using hermetic ceramic-metal feedthroughs put in through the lower sheet 11, in the case where the sheet is a part made of ceramic. These feedthroughs can be prepared by cosintering, as described, for example, in FIG. 2 in the abovementioned paper by A. Vanhoestenberghe et al.

A device for testing the hermeticity of the housing can be incorporated in the housing, in order to nondestructively describe the level of hermeticity of the housing manufactured. The helium tests conventionally employed to test the hermiticity of conventional housings made of titanium can be used with difficulty in the present invention as a result of the small volumes of the housing. In order to test the hermiticity of housings having small volumes (less than 50 mm³), provision has been made, in the document EP 1 533 270, to use a control element, the optical or electrical properties of which change in the presence of a reactive fluid. This document provides in particular for the use of a copper (Cu) layer in order to ensure optical monitoring. This is because in the presence of oxygen, the Cu layer oxidizes, thus modifying its optical properties. On oxidizing, the Cu layer becomes transparent (particularly in the near infrared) and it is thus possible to quantify the amount of oxygen which has entered the housing and to refer to a degree of leakage of the housing. This requires the use of a light source of known intensity and of a sensor in order to quantify either the light transmitted through the assembly of the housing (which requires that the housing be completely transparent) or the reflected radiation. It is thus advantageous, in the case of the present invention, to use a sensor present within the housing in order to characterize the change in the optical properties of the control element set down, for example, on the interior face of a sheet made of ceramic or of glass. It will thus be possible to translate an optical variation in the layer into an electrical signal via the sensor present within the housing. This sensor can, for example, be a photodetector or a photovoltaic cell.

1. A hermetic housing comprising:
a first element in the form of a sheet (11, 61), made of at least one material chosen from a metal, a ceramic and a glass;
a second element (15, 16; 65) having dimensions and a shape suitable for covering said first element, also made of at least one material chosen from a metal, a ceramic and a glass;
a first metal surround (12, 62) interposed between said first element and said second element, comprising an internal part (14, 64) positioned partially or completely inside the perimeter of said first element and an external part (13, 63) positioned completely outside said perimeter of said first element;
a first hermetic seal (31) between said first element and said internal part of said first metal surround; and
a second hermetic seal between said second element and said external part of said first metal surround.

2. The hermetic housing as claimed in claim 1, in which said first hermetic seal is produced by a technique chosen from: soldering, solid state diffusion welding and, when said first element is at least partially made of ceramic, cosintering.

3. The hermetic housing as claimed in claim 1, in which said second hermetic seal is produced by welding.

4. The hermetic housing as claimed in claim 1, in which said second element comprises:
an “upper” sheet (15), made of at least one material chosen from a metal, a ceramic and a glass;
a second metal surround (16) interposed between said upper sheet and said first metal surround, comprising an internal part (18) positioned partially or completely inside the perimeter of said upper sheet and an external part (17) positioned completely outside said perimeter of said upper sheet; and
a third hermetic seal (32) between said upper sheet and said internal part of said second metal surround;
said second hermetic seal being between the external parts of said first and second metal surrounds.

5. The hermetic housing as claimed in claim 4, in which said third hermetic seal is produced by a technique chosen from: soldering, solid state diffusion welding and, when said upper sheet is at least partially made of ceramic, cosintering.

6. The hermetic housing as claimed in claim 1, in which said second element comprises an “upper” sheet (65) made of metal, directly attached to the external part of said first metal surround via said second hermetic seal.

7. The hermetic housing as claimed in claim 1, in which the external part of said or of each metal surround exhibits, with respect to the corresponding internal part, an excess thickness over its face opposite said second hermetic seal.

8. The hermetic housing as claimed in claim 1, exhibiting a thickness of less than or equal to 3 mm and preferably of less than or equal to 1 mm, more preferably of less than or equal to 500 μm.

9. The hermetic housing as claimed in claim 1, comprising at least one component (41, 93) mounted on said first element in the form of a sheet, directly or via a dielectric layer, and contained in a space delimited by said first element, said second element and said metal surround or surrounds.

10. The hermetic housing as claimed in claim 9, in which said or each component is covered with a layer made of polymer material (42, 92).

11. The hermetic housing as claimed in claim 9, comprising at least one conductive track (53) arranged on said first element in the form of a sheet, said or each conductive track extending over both faces and over the side face of said first element and being covered with an insulating material (54, 55), except at its ends, forming lands.

12. The hermetic housing as claimed in claim 1, partially or completely coated with a layer made of polymer material (43, 94).

13. The hermetic housing as claimed in claim 4, in which the internal part of the upper sheet (15, 65), which is preferably metallic, is concave, in particular in order to define a cavity.

14. The hermetic housing as claimed in claim 13, in which the external part of the upper sheet (15, 65), which is preferably metallic, is also concave.

15. The hermetic housing as claimed in claim 1, in which the first element in the form of a sheet (11, 61) exhibits a U shape, in particular in order to define a cavity.

16. The hermetic housing as claimed in claim 1, comprising:
a first element in the form of a sheet (11, 61) made of a ceramic;
an additional surround (132, 142) also made of ceramic; and
one or more metal electrodes (131, 141), the or each metal electrode (131, 141) exhibiting a portion (1313) sandwiched between the first element in the form of a sheet (11, 61) and the additional surround made of ceramic (132, 142), a first end (1311, 1411) accessible on an upper face (52) of the first element in the form of a sheet (11, 61) and a second end (1312, 1412) accessible either on a lower face (56) of the first element in the form of a sheet (11, 61) or on a lower face (1422) of the additional surround made of ceramic (142).

17. The housing as claimed in claim 16, in which a hermetic seal is provided between the internal part (14, 64) of the first metal surround (12, 62) and the additional surround made of ceramic (132, 142).

18. The hermetic housing as claimed in claim 1, comprising:
a first element in the form of a sheet (11, 61) made of metal;
a first additional surround (152) made of ceramic;
a second additional surround (153), also made of ceramic;
one or more metal electrodes (151), the or each metal electrode (151) exhibiting a portion (1513) sandwiched between the first additional surround made of ceramic (152) and the second additional surround made of ceramic (153), a first end (1511) accessible on an upper face (1521) of the first additional surround made of ceramic (152) and a second end (1512) accessible on a lower face (1532) of the second additional surround made of ceramic (153).

19. The hermetic housing as claimed in claim 18, in which a hermetic seal is provided between the internal part of the first metal surround (12, 62) and the second additional surround made of ceramic (153).

20. The hermetic housing as claimed in claim 18, in which a hermetic seal is provided between the upper face (52) of the first element in the form of a sheet (11, 61) and the lower face (1522) of the first additional surround made of ceramic (152).

21. The hermetic housing as claimed in claim 1, exhibiting the shape of a cross (122) so that it can be curved along several axes, in particular axes which are non parallel with one another.

22. The hermetic housing as claimed in claim 1, at least partially made of biocompatible material or materials, so as to be implantable in a human or animal body.

23. A device comprising several housings as claimed in claim 1, characterized in that said housings are connected mechanically to one another by a flexible common support (124), for example made of polymer, in order to improve the flexibility of the assembly thus formed by said housings.

24. The device as claimed in claim 23, in which the flexible common support (124) is adhesively bonded to the housings or overmolded, partially or completely, over said housings.

25. The device as claimed in claim 23, in which the flexible common support (124) comprises metal tracks or wires in order to electrically connect housings to one another.

26. The device as claimed in claim 23, in which the flexible common support (124) comprises one or more electronic components, for example an antenna.

27. The device as claimed in claim 23, the housings of which are arranged in order to confer a cross shape on said device, so that the latter can be curved along several axes, in particular axes which are non parallel with one another.

28. An implantable medical device comprising a hermetic housing as claimed in claim 22.

29. A process for the encapsulation of a device, comprising the stages consisting in:
mounting at least one component (41, 93) of said device to be encapsulated on a first element in the form of a sheet (11, 61), made of at least one material chosen from a metal, a ceramic and a glass;
superimposing, on said first element in the form of a sheet, a first metal surround (12, 62) comprising an internal part (14, 64) positioned partially or completely inside the perimeter of said first element and an external part (13, 63) positioned completely outside said perimeter of said first element;
forming a first hermetic seal (31) between said first element and said internal part of said first metal surround;
superimposing, on said first metal surround, a second element (15, 16; 65) with dimensions and a shape suitable for covering said first element, also made of at least one material chosen from a metal, a ceramic and a glass; and
forming a second hermetic seal between said second element and said external part of said first metal surround.

30. The process as claimed in claim 29, in which the stage of formation of said first hermetic seal is carried out by a technique chosen from: soldering, solid state diffusion welding and, when said second element is at least partially made of ceramic, cosintering.

31. The process as claimed in claim 29, in which the stage of formation of said second hermetic seal is carried out by welding.

32. The process as claimed in claim 29, in which said second element comprises:
an “upper” sheet (15), made of at least one material chosen from a metal, a ceramic and a glass;
a second metal surround (16) interposed between said upper sheet and said first metal surround and comprising an internal part (18) positioned partially or completely inside the perimeter of said upper sheet and an external part (17) positioned completely outside said perimeter of said upper sheet;
the process also comprising a stage of formation of a third hermetic seal (32) between said upper sheet and said internal part of said second metal surround, said stage being carried out before the formation of said second hermetic seal between the external parts of said first and second metal surrounds.

33. The process as claimed in claim 32, in which the stage of formation of said third hermetic seal is carried out by a technique chosen from: soldering, solid state diffusion welding and, when said upper sheet is at least partially made of ceramic, cosintering.

34. The process as claimed in claim 29, in which said second element comprises an “upper” sheet (65) made of metal and in which said second hermetic seal is formed directly between said upper sheet and the external part of said first metal surround.

35. An implantable medical device comprising the device as claimed in claim 23.
